(19) United States
(12) Patent Application Publication
(10) Pub. No.: US 2008/0262550 A1
(43) Pub. Date: Oct. 23, 2008

(54) CROSS-COUPLED VERTEBRAL STABILIZERS INCORPORATING SPINAL MOTION RESTRICTION

(76) Inventor: Bret A. Ferree, Cincinnati, OH (US)

Correspondence Address: O''Melveny & Myers LLP;IP&T Calendar Department LA-1118, 400 South Hope Street, Los Angeles, CA, 90071-2899, US

(21) Appl. No.: 12/165,321

(22) Filed: Jun. 30, 2008

Related U.S. Application Data
(63) Continuation of application No. 10/152,485, filed on May 21, 2002.
Application No. 10/152,485 is a continuation-in-part of application No. 09/841,324, filed on Apr. 24, 2001, now Pat. No. 6,423,065.
Application No. 09/841,324 is a continuation-in-part of application No. 09/513,127, filed on Feb. 25, 2000, now Pat. No. 6,248,106.

(51) Int. Cl. A61B 17/70; A61B 17/04

(52) U.S. Cl. 606/263; 606/246; 606/301

(57) ABSTRACT

Methods for stabilizing upper and lower spinal vertebrae having a disc situated between the upper and lower vertebrae are described. First and second fasteners are inserted into the upper vertebra. Third and fourth fasteners are inserted into the lower vertebra. At least two of the first, second, third, and fourth fasteners are connected with an elongate element. In an alternative embodiment, at least three of the first, second, third, and fourth fasteners are connected with the elongate element. The elongate element may be an elastic connector or a cable. The elongate element may also have first and second ends that are connected by a crimp.

### REFERENCE TO RELATED APPLICATION

This application is a continuation of U.S. patent application Ser. No. 10/152,485, filed May 21, 2002, which is a continuation-in-part of U.S. patent application Ser. No. 09/841,324, filed Apr. 24, 2001, which is a continuation-in-part of U.S. patent application Ser. No. 09/513,127, filed Feb. 25, 2000, now U.S. Pat. No. 6,248,106, the entire content of each application being incorporated herein by reference.

### FIELD OF THE INVENTION

This invention relates generally to orthopedic spinal surgery and, in particular, to vertebral fixation methods and apparatus which provide multi-dimensional stability and apply compressive forces to enhance fusion.

### BACKGROUND OF THE INVENTION

In surgeries involving spinal fixation, interbody cages are often used to restore disc space height, serve as a conduit for bone graft, and to help immobilize vertebrae undergoing fusion. Distracting the disc space prior to cage insertion restore disc space height. Distraction serves two important functions. First, it can decrease pressure on spinal nerves by increasing the size of the intervertebral foramen. Second, distraction increases tension on the annulus fibrosis which, in turn, increases the stability of the vertebra-cage-vertebra construct.

Presumably the annular tension decreases with time, thus weakening the construct. Furthermore, the annulus is weakened in many patients with severe degenerative disc disease. Given these and other deficiencies with annular tension, additional fixation is frequently added to increase the rigidity of the vertebra-cage combination.

Currently such additional fixation is inserted onto or into the posterior aspect of the spine. Thus, patients who have cages inserted from an anterior approach must undergo a second operation from the posterior aspect of the body. As might be expected, the second surgery increases patient morbidity, insurance costs, and delays return to work.

There are two ways to insert supplemental fixation through the same incision. One technique uses the interbody cages disclosed in my co-pending U.S. patent application Ser. No. 09/454,908, the entire contents of which are incorporated herein by reference. Posterior insertion allows the addition of supplemental fixation through the same incision.

A second solution employs fixation inserted through the anterior aspect of the spine. With known anterior lumbar spine fixation techniques, a combination of screws and rods or plates are inserted on the lateral side of the vertebrae from an anterior or lateral approach. The fixation is placed on the lateral aspect of the spine to avoid the aorta. Previous metal devices placed under the aorta have lead to aneurysms in some cases (Dunn Device). Unfortunately, a few patients have died from rupture of the aneurysms.

Lateral fixation is not ideal with interbody cages. First, lateral fixation cannot be used at the L5-S1 level. The iliac arteries cross the L5-S1 level anteriorly and laterally. Second, the vascular anatomy of many patients does not permit lateral fixation at the L4-L5 level. The majority of cages are inserted at the L4-L5 and L5-S1 levels. Third, cages are generally inserted in a directly anterior-to-posterior fashion with the patient in a supine position. Lateral instrumentation is difficult if not impossible in most patients in the supine position.

The system described in U.S. Pat. No. 5,904,682 uses two flat plates applied to screws placed bilaterally on either side of the disc space. The system does not use cables or diagonal bracing to resist rotational forces. In U.S. Pat. No. 4,854,304 screws laced in the side of the vertebral bodies are connected from a lateral approach. The screws are connected with a threaded rod. In 1964, A. F. Dwyer described a system using a single cable to connect screws placed on the lateral portion of the vertebral bodies. Dr. Dwyer connected a series of screws with one screw per vertebral body. The arrangement described in U.S. Pat. No. 4,854,304 is similar to Dr. Dwyer's system, but the cable is replaced with a threaded rod. Dr. Ziekle modified Dr. Dwyer's system in 1975, as set forth in U.S. Pat. No. 4,854,304.

Cables and tensioning devices are also well known in orthopedic spine surgery. References that use cables include U.S. Pat. No. 4,966,600; 5,423,820; 5,611,801; 5,702,399; 5,964,769; 5,997,542. None use diagonal members to enhance compression and resist lateral movement, however.

### SUMMARY OF THE INVENTION

My U.S. Pat. No. 6,248,106 is directed to spinal stabilization mechanisms operative to prevent lateral bending, extension, and rotation at the disc space. Broadly, the mechanism includes two or more anchors at each vertebral level, and links for each anchor at each level to both anchors at the other level, resulting in a cross-braced arrangement.

In the preferred embodiment, the mechanism uses screws for placement in the vertebral bodies and cables are used to connect the screws. The cables pull the screws together, applying compression across the disc space. Bone graft, cages, or distracting plugs and the device to enhance fusion area would fill or cross the disc space. The bone graft, cages, etc. within the disc space are preferably used to resist compression.

The device may be used in the cervical, thoracic, or lumbar spine. The device is preferably placed anteriorly, but could also be used posteriorly, with the screws directed through the vertebral body pedicles. The various components may be constructed of titanium, stainless steel, polymers, or a combination of such materials.

The anchors preferably include a post protruding from the vertebra, and a cable-holders which fits over the post. The post may be threaded, in which case a nut would be used to tighten the holders, or the cable holders may be allowed to rotate, depending upon the position and/or application of the fasteners. The cable holders may use tunnels, tubes or outer grooves to the hold the cables in position. Devices may also be added to keep the links from crossing one another where they cross.

My U.S. patent application Ser. No. 09/841,324 discloses a refinement comprising a cam-operated cable-holding connector which may be used for vertebral alignment and other applications. The connector includes a lower screw portion configured to penetrate into a vertebrae, thereby leaving an exposed portion. A cable-holding mechanism attached to the exposed portion is operable between a first state, wherein one or more cables may be readily dressed therepast, and a second state, wherein a portion of the mechanism is rotated or otherwise physically manipulated to lock the one or more of the cables into position.

In the case of vertebral alignment, the lower screw portion is preferably a pedicle screw, and the mechanism includes a first body having an interrupted side wall with an inner surface, and a second body having a rotatable cam. In this case, the mechanism facilitates a first state, wherein the relationship between the cam and the inner surface of the side wall is such that the cables pass therethrough, and a second state, wherein the cam is turned so as to retain the one or more cables against the inner wall of the side wall.

Pedicle screws are generally connected by solid rods or plates in an attempt to eliminate spinal motion. Eliminating spinal motion helps the vertebrae fuse together. A few inventors have connected pedicle screws with rubber, elastic, or fibrous materials to dampen or restrict spinal motion. These inventors have postulated low back pain is caused by abnormal movements and/or pressure across the facet joints.

Initially, the pedicle screws were connected by fibrous bands to limit flexion of the spine (distraction of the posterior portion of the vertebrae). The devices were improved by covering the fibrous bands with rubber sleeves which help dampen the forces on the facets that occurs with spinal extension. That is, the rubber sleeves help prevent extension of the spine. Forces on the facets increase with extension.

Lumbar facet joints also restrict twisting of the spine. Naturally, the force on the facet joints also increases with twisting or rotation of the spine. The prior-art devices do not dampen the rotational forces applied to the spine. Thus, low back pain from rotational forces on arthritic facet joints is not prevented with prior art devices.

This invention improves upon the prior art through the addition of cross-coupled members to help prevent rotational forces on the facet joints, with particular emphasis on the posterior portion of the lumbar spine. Rigid, semi-rigid, or elastic members may be used depending upon the desired degree of resistance.

The cross-coupled members may assume different forms, including cables and polymer, fibrous, or elastic bands. For example, vertebral motion may be damped by connecting the screws with elastic bands. Vertebral motion could be further damped by covering the anterior bands with rubber or elastomeric sleeves similar to the sleeves used over the posterior bands of the prior art devices described above.

Although the configuration may be used as an adjunct to spinal fusion, it may also be used to dampen motion as an adjunct to vertebral anthroplasty.

### BRIEF DESCRIPTION OF THE DRAWINGS

FIG. 1A is an anterior view of a cable-based cross-coupled vertebral stabilizing mechanism according to U.S. Pat. No. 6,248,106;

FIG. 1B is a drawing which shows the mechanism of FIG. 1A from a lateral perspective;

FIG. 2 is a drawing which shows how cable-receiving discs may be stacked to join three or more vertebrae;

FIG. 3 is a drawing which shows how different types of cable-holding devices may be combined to join multiple vertebra;

FIG. 4 shows the use of preformed sleeves;

FIG. 5 depicts the use of additional devices for protecting cables from abrading one another where they cross;

FIG. 6 is a drawing which illustrates the alternative use of a centerpiece with four cables attached thereto using screws or alternative fasteners;

FIG. 7 is a drawing which illustrates the alternative use of turnbuckles on one or more cables;

FIG. 8 is a view in perspective of different elements constituting a stabilization device according to U.S. Pat. No. 5,540,688, to which the instant invention is applicable;

FIG. 9 is a view from behind of three vertebrae associated with the stabilization devices of FIG. 8;

FIG. 10 is a section along III-III of FIG. 9;

FIG. 11 is a posterior view of a prior-art vertebral stabilizing mechanism including cross-coupled stabilization according to the invention;

FIG. 12 illustrates an attachment arrangement other than pedicle screws.

FIG. 13 is an oblique, exploded-view drawing of a cable holding and tensioner according to the invention;

FIG. 14 is a drawing which shows a tool adapted to insert fasteners and tighten cable holders according to the invention;

FIG. 15A is an oblique drawing of one type of blocking cable-receiving disc according to the invention;

FIG. 15B is an oblique drawing of an alternative cable-receiving locking disc according to the invention;

FIG. 15C illustrates yet a further alternative cable-receiving disc according to the invention;

FIG. 16 is a drawing which shows how cable-receiving discs of the type shown in FIG. 15C allows a cable to move through a range of angles;

FIG. 17 shows how cable holders according to the invention may rotate around a respective post, in which case a tightening nut may not be necessary;

FIG. 18 illustrates how a single crimp may be used instead of separate crimping at each cable holder;

FIG. 19 is a drawing which shows an alternative fastener according to the invention affording an enhanced range of cable motion;

FIG. 20 is a drawing which shows how multiple cables or bands may be dressed from one fastener to another for enhanced security;

FIG. 21 is a drawing which shows how structures according to the invention may be used to correct spinal deformities such as scoliosis;

FIGS. 22A-22F are drawings of a further alternative embodiment of the invention including a mechanism which locks on or more cables into position. In particular,

FIG. 22A is a side-view of a connector including a rotating cable lock;

FIG. 22B shows the device of FIG. 22A in an exploded view, part of which is in cross-section;

FIG. 22C is a top view of the lower portion of a cable-locking body;

FIG. 22D is an on-axis view of the top portion of a cable-locking body;

FIG. 22E is a top down view revealing the first state of the mechanism of FIGS. 22A-22D wherein two cables have been threaded therethrough prior to locking; and

FIG. 22F is a drawing of the arrangement of FIG. 22E, having been rotated to lock the two cables into place.

### DETAILED DESCRIPTION OF THE INVENTION

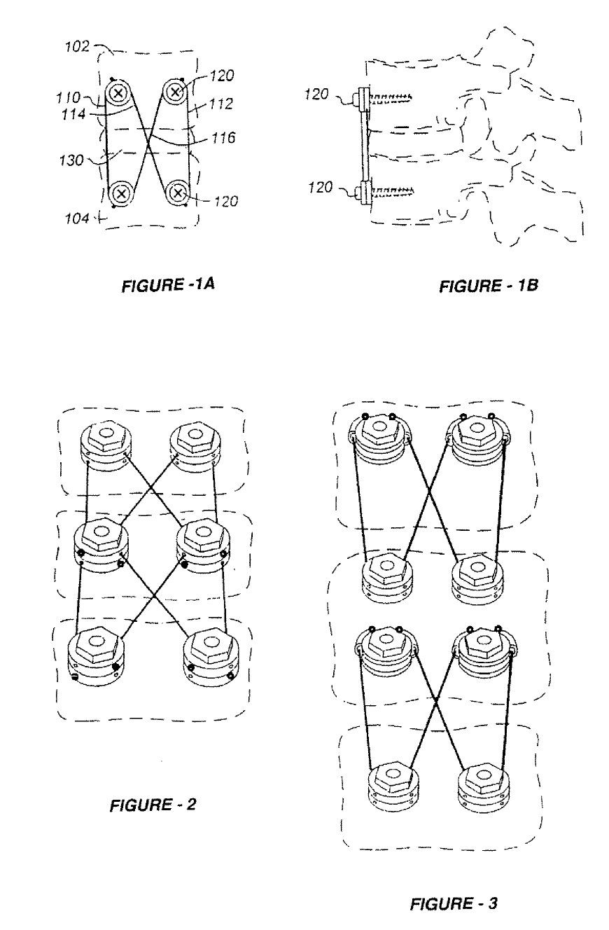

FIG. 1A is an anterior view of a cable-based cross-coupled vertebral stabilizing mechanism disclosed in U.S. Pat. No. 6,248,106, incorporated herein by reference. FIG. 1B is a drawing which shows the mechanism of FIG. 1A from a lateral perspective. In this illustration, the mechanism is used to join upper and lower vertebrae 102 and 104, respectively, though the mechanism is applicable to multiple levels, as shown in FIGS. 2 and 3. Note that some form of intervertebral cage and/or bone graft 130 may be used in between the vertebrae 102 and 104 to resist compression.

Broadly, the mechanism utilizes a pair of fasteners on each vertebrae, and elongated elements, preferably cables, in an axial and criss-crossed pattern to provide an arrangement that resists extension, lateral bending, and torsional/rotational stresses. As best seen in FIG. 1A, a preferred configuration utilizes a pair of screws 120 in the upper vertebrae, and a corresponding pair in the lower vertebrae, along with a pair of longitudinal cables 110 and 112, which are used in conjunction with a pair of criss-cross cables 114 and 116.

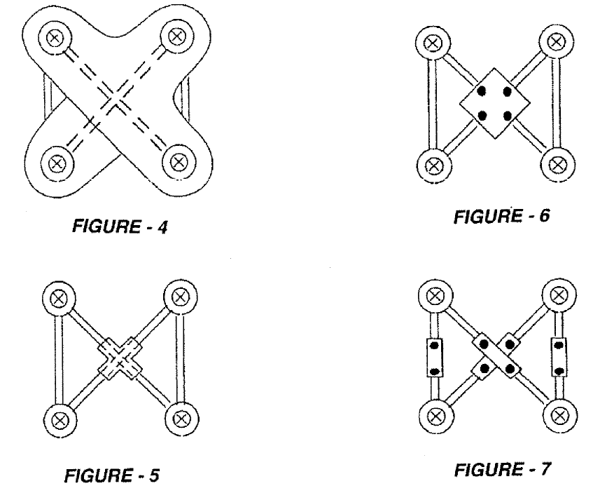

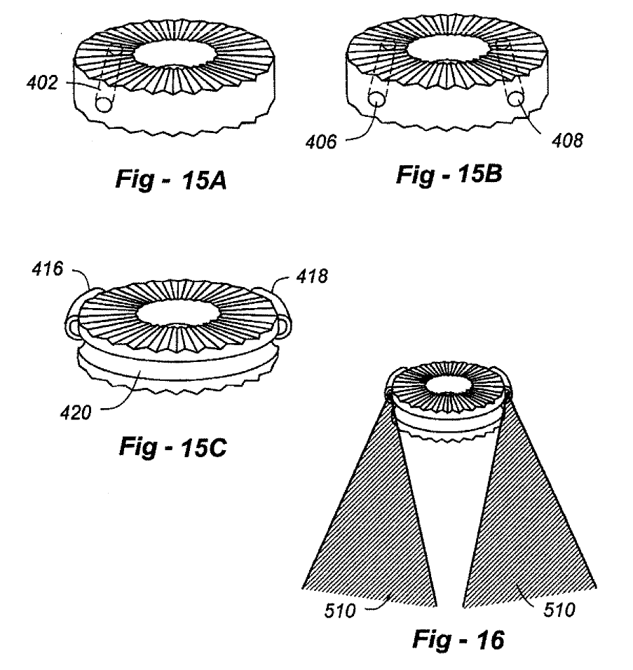

FIG. 2 is a drawing which shows how cable-receiving discs of the type may be stacked to join three or more vertebrae. FIG. 3 shows how different types of cable-holding devices may be combined to join multiple vertebra. In this particular case, devices of type FIG. 15C are combined with those of FIG. 15A or 15B, with middle or intermediate vertebra (702) incorporating two sets fasteners. Such devices may be covered with soft materials such as silastic in various ways. For example, preformed sleeves may be placed over prominent portions of the device, as shown in FIG. 4. Alternatively, liquid polymer may be poured over, or injected to surround the device. The material could be strengthened by inserting fibers into and around the device before or during the pouring or injection procedure. Polymer would be selected on the basis that it would cure rapidly and safely within the body.

Additional devices may be provided to protect the cables from abrading one another where they cross in the middle. For example, an x-shaped device with holes could be placed over the crossing wires, as shown in FIG. 5. Preferably, the wires would cross over the device in different planes to prevent friction with one another. Alternatively, a centerpiece could be used wherein four cables attached thereto using screws or alternative fasteners (FIG. 6). As yet a further alternative, as shown in FIG. 7, turnbuckles may be incorporated into the cables or threaded rods to tighten them during installation or, perhaps as part of a postoperative or revision procedure.

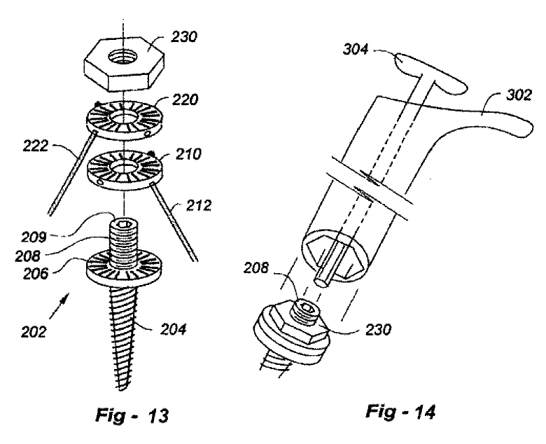

The invention anticipates various apparatus for holding and tightening the cables or alternative members. FIG. 13 is an oblique, exploded-view drawing of a cable holding and tensioner according to the invention. In this configuration, a screw 202 features a threaded end 204, an exposed seating surface 206 and a threaded post 208, preferably including a tool receiving aperture 209 described in more detail below. A plurality of discs 210 and 220, each receiving a cable 212 and 222, respectively, are stacked onto the threaded post 208 and a nut 230 is tightened thereon as shown in the drawing, the seating surface 206, as well as the opposing surfaces of the discs 210 and 220 preferably include radial grooves or an alternative form of surface pattern or texture operative to resist rotation when the discs are stacked on top of one another and the fastener 230 tightened.

FIG. 14 is a drawing which shows a tool adapted to insert fasteners and tighten cable holders according to the invention. Such a tool includes two elongated, independently rotating portions, including a nut tightening wrench 302 which fits over the locking nut 230 shown in FIG. 13, and a hex head screwdriver 304 which fits into the aperture 209 to prevent the screw from rotating while tightening the nut 230.

As mentioned above, the invention preferably utilizes different types of cable-receiving discs, depending upon placement and tensioning procedure. FIG. 15A is an oblique drawing of one type of blocking cable-receiving disc according to the invention. This particular disc includes a single aperture 402 through one side of the disc, which would require multiple discs to be stacked on each threaded post in a crisscross tensioning configuration. FIG. 15B is an oblique drawing of an alternative cable-receiving locking disc according to the invention. In this configuration, double cable-receiving apertures 406 and 408 are provided, preferably oriented along non-parallel lines, as shown. As in the case of the disc of FIG. 15A, the device would simply be turned over to provide a different desired cable orientation. FIG. 15C illustrates yet a further alternative cable-receiving disc according to the invention. With the device of FIG. 15C, one or more tubes 416 and 418 are provided, preferably along with a cable-receiving groove 420. As shown in FIG. 16, the device of FIG. 15C allows a cable to move through a range of angles 510 without creating an area with an acute bend or stress riser.

The cable-receiving disc of FIG. 15B would be used in the same manner as that of FIG. 15A, though it should be evident that only one disc is required per fastener, as opposed to two. However, by stacking the cable-receiving discs of the type shown in FIG. 15B, more than two vertebrae levels may be joined with a minimum number of devices, and/or additional cables may be provided for added stability.

Those of skill in the art of orthopedic surgery will appreciate that certain of the tools and techniques used to tighten and secure the cable-holding bodies are known, and therefore do not necessarily form part of this invention. For example, tools of the type shown in FIG. 14 are available for related purposes, and may be used for the inventive purposes disclosed herein, perhaps with minor modification, as appropriate. In addition, known cable pulling/tensioning tools and cutting and crimping techniques used in orthopedic surgery may also be applied to the instant invention, again, with any necessary modifications, as appropriate, to accommodate the physiology associated with the spine or particular level of the vertebrae.

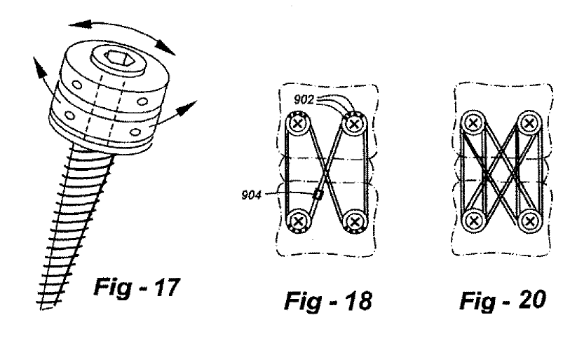

Although the cable-holding bodies of FIGS. 15A-15C preferably feature a friction-increasing upper and lower surfaces to resist rotation when tightened down, the cable holders may, instead, rotate around the post, as shown in FIG. 17, in which case a tightening nut would not be necessary. As a further alternative, the cables may be wound around a different type of holder similar to that in FIG. 15C, but without the cable-holding tubes. In this alternative embodiment, the grooves in the holder, which may or may not rotate, would include multiple cable-retainers 902, enabling a single crimp 904 to be used instead of separate crimping at each cable holder.

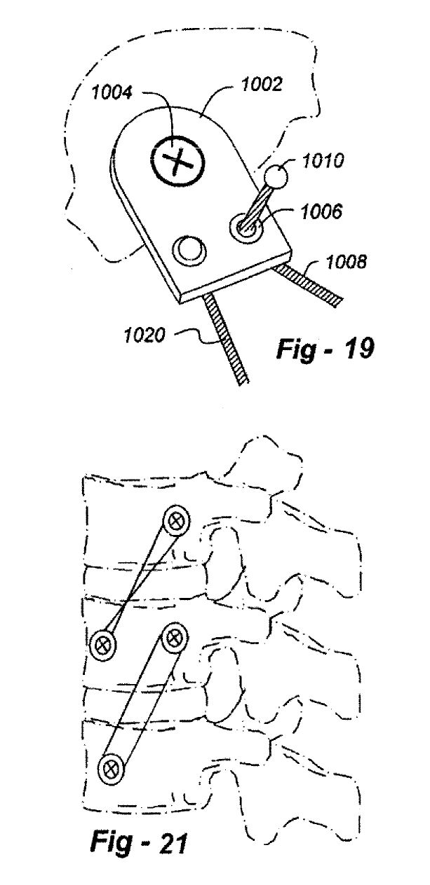

FIG. 19 is a drawing which shows an alternative cable holder according to the invention having a body 1002 attached to bone using a fastener 1004. One or more cable-receiving holes are provided and, in this case, the holes include cup-shaped recesses 1006. With such a configuration, a cable 1008 having a hall-shaped end 1010 is received by the recess, allowing the cable to more from side to side through rotation of the ball within the cup-shaped recess. Cable 1020 is shown having its ball-shaped end seated in the recess. As an alternative to a pre-formed ball an appropriately shaped crimp would also provide for an improved range of cable motion.

Multiple cables or elastic connectors may also be dressed from one fastener to another for enhanced stability, as shown in FIG. 20. For example, the holders of FIG. 15B may be stacked on each threaded post, enabling two cables to run from each fastener at each level. Alternatively, multiple holders of the type shown in FIG. 15C, or those shown in FIG. 18 may be stacked on each fastener post or, alternatively, a groove may be provided having a width sufficient to accommodate multiple cables or bands, and this may be permitted to rotate or lock into place, depending upon the situation at hand or preference of the surgeon.

To prevent injury to surrounding structures such as the aorta, devices according to the invention may be covered with a soft material such as siliastic. Fixation devices placed on the anterior aspect of the spine risk erosion into the aorta in the thoracic and lumbar spine regions, and in the esophagus in the cervical spine. The metal from plates or screws is unyielding, and as the aorta pulses into the metal a hole can form in the wall of the vessel. Discs may also herniated and anteriorily. In addition, bone spurs from the vertebrae can project anteriorily. At times, both this material and bone spurs may press against the aorta. This natural process does not injure the aorta or the esophagus, presumably because the soft disc material yields to the pulsations of aorta. Bone spurs probably reabsorb if they are causing injury to surrounding structures.

The mechanisms described in the various embodiments of the invention offer several advantages over existing devices. The first, in contrast to current devices which do not permit compression, the inventive structure applies compression across the disc space. Compression is thought to increase the chances of a successful fusion. The inventive mechanism also allows the vertebrae to come together if the graft and or cage collapses or subsides; that is falls deeper into the body of the vertebrae. Many current devices hold the vertebrae apart when the graft collapses, which increases the chances of a pseudoarthrosis.

The inventive mechanism has a low profile, which may often allow placement under the aorta. A low profile is also beneficial in the cervical region of the spine. The inventive mechanism may also provide supplemental fixation within the body cages, which would increase the rigidity of the cage construct. Furthermore, devices according to the invention maintain compression across the disc space when the annular tension fails. As such, the inventive structures obviate a second, posterior operation to place screws and rods over the vertebrae.

Exact screw placement is made easier by virtue of the invention. Often screws are directed through plates placed on the spine, which make screw placement imprecise, leading to misdirected screws into adjacent disc spaces or laterally into the vertebrae. The device also affords the possibility of flexibility in patients with spinal deformities. As shown in FIG. 21, structures according to the invention may also be used to correct spinal deformities. For example, rotation of vertebrae would be more easily corrected using cables and cable holding devices according to the invention, in contrast to currently available appliances. Used on the sides of the vertebrae the cabling would correct for rotational deformities and easy insertion of current devices, should a surgeon wish to add rods and/or screws. In terms of the correction of spinal deformities, one could tighten the cable, or set of cables, to first to correct for scoliosis.

Another advantage is that additional levels of the spine may be added in subsequent surgeries without dismantling the entire device. That is, holding bolts may be removed, and new cable-holding bodies added, or, with grooves wide enough to permit multiple cables, new cabling alone may be added to multiple levels. The inventive mechanisms help hold in bone graft, cages or other devices to enhance fusion, while not stressing the “shield” of the bone graft.

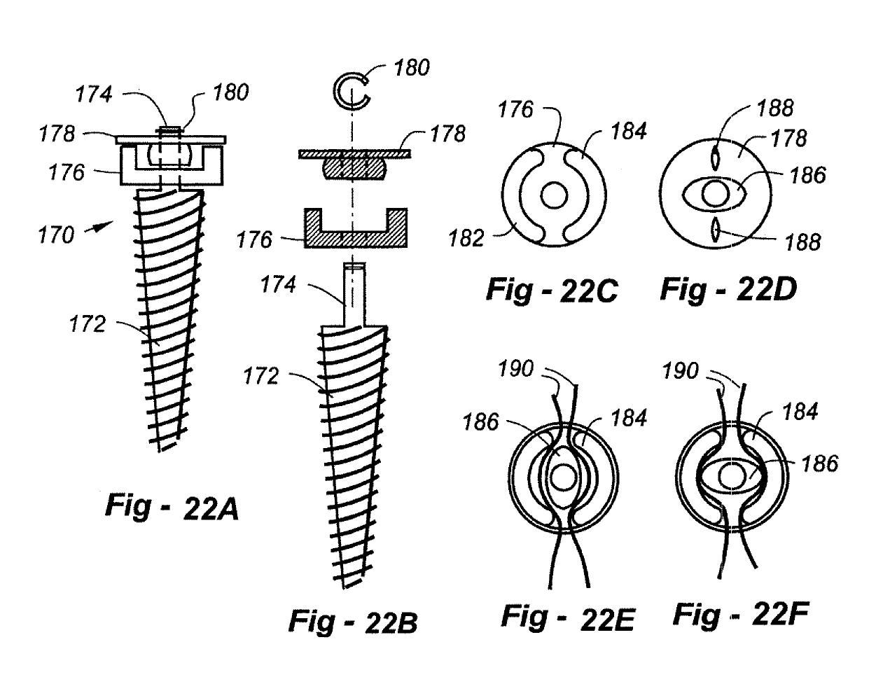

FIGS. 22A-22F provide different views of a different embodiment of the invention including the rotatable cable lock assembly. FIG. 22A is a side view of the embodiment depicted generally at 170. The configuration preferably includes a pedicle screw portion 172, terminating upwardly in a post 174. Onto the post 174, there is journaled a lower portion 176 of a locking mechanism, and an upper, rotatable portion 178. As better seen in FIGS. 22C-22F, the lower and upper portions 176 and 178 include a central bore through which the post 174 on pedicle screw 172 extends, the post including a circumferential groove to receive a fastener such as C-clip 180. Other devices, such as cotter pins, flared ends, and the like, may alternative be used instead of the C-clip 180.

FIG. 22B is a side view of the device of FIG. 22A in an exploded view, with at least the lower portion 176 and upper 178 being depicted in cross-section. FIG. 22C is an on-axis view of the lower portion 176, where it can be seen that the component includes raised circular side walls 182 and 184 to receive a cam 186 in rotating engagement. This cam 186 is integrally formed with the upper component 178, as shown in FIG. 22B. The arrangement further includes a pair of slots 188 or other features to engage with a tool (not shown) to rotate the member 178 and cam 186 to lock one or more cables into position.

This action is depicted in FIGS. 22E and 22F. In FIG. 22E, the cam 186 is aligned between the openings of cylindrical side walls 182 and 184, enabling cables 190 to be threaded therethrough. Having fed the one or more cables 190 through the locking mechanism, the upper portion 178 is turned through the interaction of a tool and features such as slots 188, thereby pinching the cables 190 between the tips of the cam 186 and the inner side walls of the raised portions 182 and 184. In the preferred embodiment, the geometry of the upper and lower portions 176 and 178, as well as the diameter and compressibility of the cables 190 are selected such that with the tips of the cam portion now perpendicular to the openings between the side walls, the cables are pinched and locked into position, preventing further rotation, but preferably without compromising the strength or holding power of the cables.

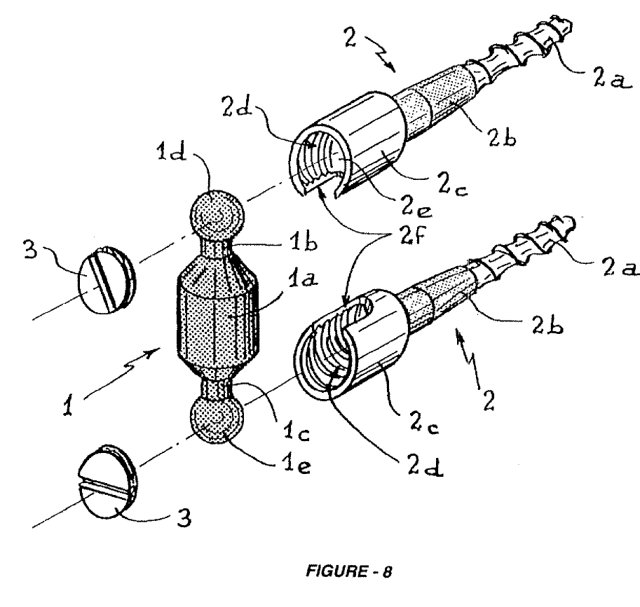

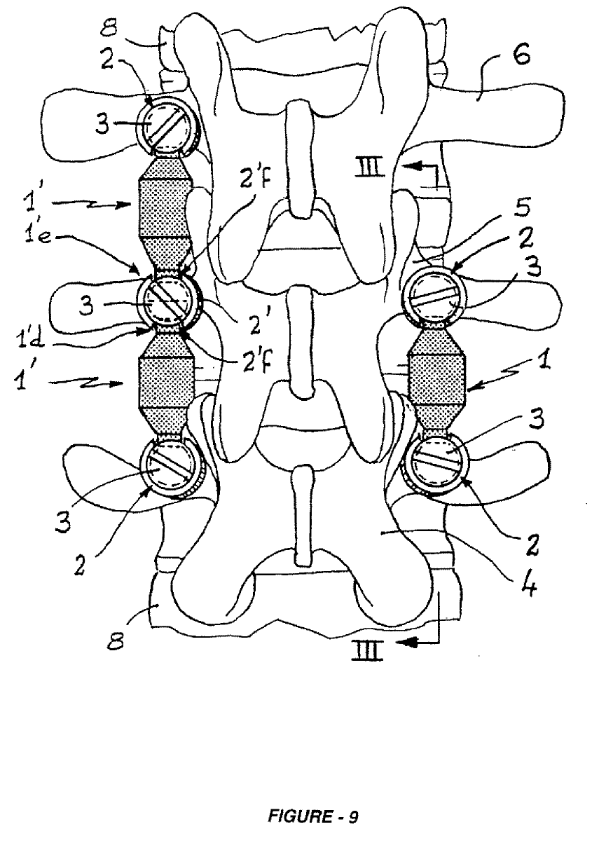

FIG. 8 is a view in perspective of different elements constituting a stabilization device according to U.S. Pat. No. 5,540,688, the entire content of which is incorporated herein by reference. The instant invention is applicable this device as well as to any other apparatus which provides two or more spinally aligned intervertebral stabilization devices, particularly those installed using pedicle screws and including dampers, as disclosed in U.S. Pat. Nos. 5,375,823; 5,480,401; 5,584,834; 5,591,166; 5,628,740; 5,961,516; EP 576379; EP 611554; EP 667127, and FR 2697428, all of which are incorporated herein by reference.

The device of U.S. Pat. No. 5,540,688 essentially comprises a damper 1 made of a bio-compatible, elastic material and two implants 2 screwed in two adjacent vertebrae and whose free ends are associated with the two ends of the damper 1. It is observed that the damper 1 is made in the form of an elongated body provided with a bulged or enlarged central part 1a joined to two necks 1b, 1c to two bulbous ends 1d, 1e. In an advantageous embodiment of the preceding arrangement, the bulged part 1a may be provided to be of elliptic longitudinal section, while the two ends 1d and 1c each take the form of a sphere. Of course, the part 1a may be of cylindrical section with two truncated endpieces or in the form of two frustums of cone or may be asymmetrical in particular applications.

Each implant 1 includes a screw 2a adapted to be screwed in the pedicle of a vertebra or in any other location thereof. The screw 2a extends from a cylindrical body 2b which terminates in a hollow socket or receptacle 2c of cylindrical shape with a tapped inner wall 2d and a concave bottom 2e presenting a shape complementary to that of half the end 1d, 1e of the damper. It is observed that the socket 2c is provided with a lateral notch 2f adapted to allow passage of the neck 1b, 1c of the damper 1 for positioning the damper with respect to the implants. Locking of the ends of the damper 1 is effected after they have been placed in the sockets 2c by screwing a threaded endpiece 3 inside the corresponding socket with respect to the tapped wall 2d. Of course, the base 3a of the endpiece 3 is provided to be concave and hemi-spherical, so as to cooperate exactly with the spherical ends 1d, 1e of the damper.

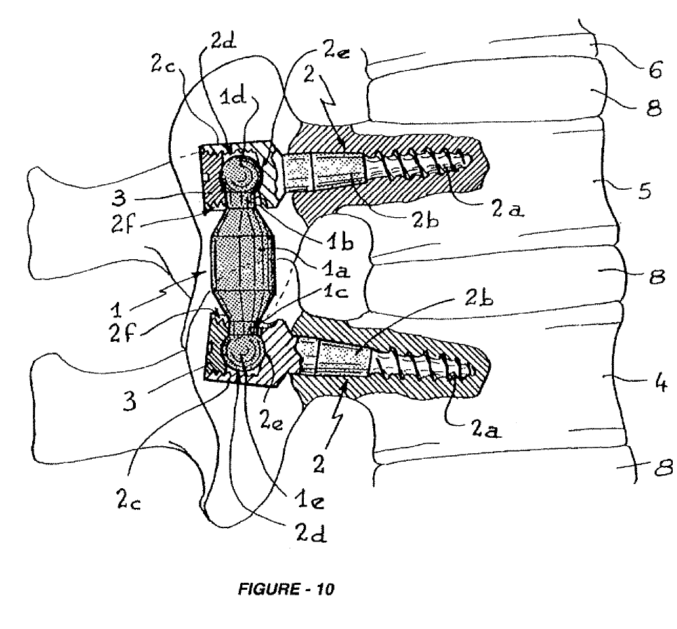

FIGS. 9 and 10 illustrate the assembly of a device according to the invention with respect to two adjacent vertebrae 4 and 5 of a spine. On the right-hand side of FIG. 9, a device has been illustrated, comprising one damper 1 associated with two implants 2 each fastened to a vertebra 4, 5. The same assembly may be provided in the left-hand part. In addition, it is possible that three successive vertebrae 4, 5, 6 need stabilization. In that case, one of the implants 2′ comprises two diametrically opposite notches 2f, while the ends of the two dampers 1′ each comprise one end 1′d, 1′e, truncated along a diametrical plane of the sphere perpendicular to the longitudinal axis of the damper in order that the two truncated ends 1′d, 1′e may be retained in the socket of the implant 2′ (cf. the left-hand part of FIG. 9).

FIG. 10 shows in very detailed manner the structure of the assembly of the ends of the damper with two implants. The hollow socket 2c with bellied concave base 2e is found again, as well as the endpiece 3 with bellied concave base 3a in order that the two spherical ends 1c, 1d of the damper 1 are suitably locked with respect to the implants 2. Such locking makes it possible to create a sort of ball joint articulation facilitating the movements of the spine.

Accordingly, prior-art devices of the type just described do not dampen the rotational forces applied to the spine. Anatomically, the lumbar facet joints restrict twisting of the spine, and the force on the facet joints increases with increasing twisting and/or rotation. Thus, low back pain from rotational forces on arthritic facet joints is not prevented with these devices.

This invention improves upon the prior art through the addition of cross-coupled members to help prevent rotational forces on the facet joints, with particular emphasis on the posterior portion of the lumbar spine. The cross-coupled members may assume different forms, including cables and polymer, fibrous, or elastic bands. Although the configuration may be used as an adjunct to spinal fusion, it may also be used to dampen motion as an adjunct to vertebral anthroplasty.

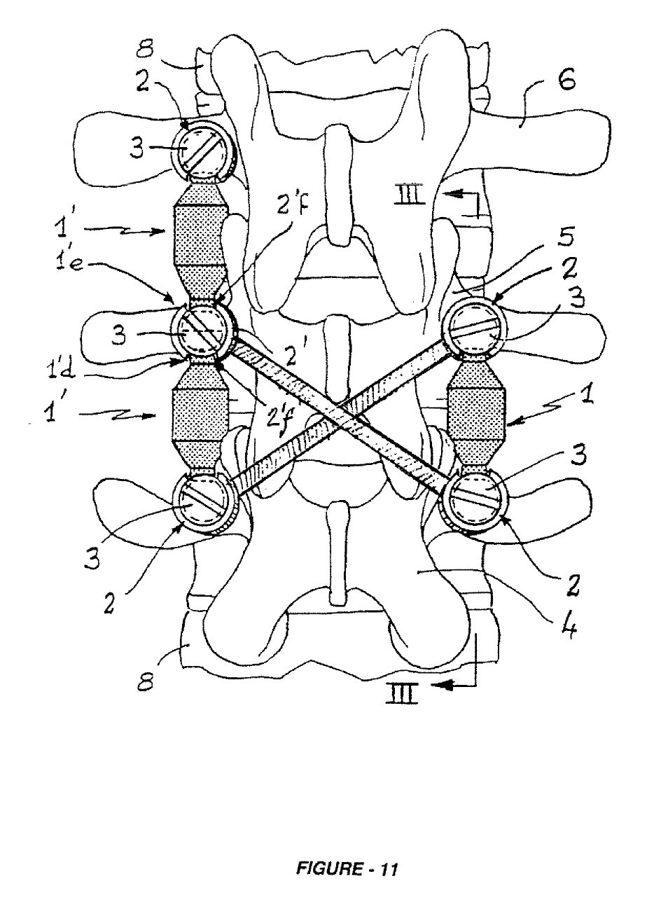

FIG. 11 is a posterior view of the prior-art vertebral stabilizing mechanism of FIGS. 8 through 10, but including cross-coupled stabilization according to this invention. Rigid, semi-rigid, or elastic members may be used depending upon the desired degree of resistance. For example, vertebral motion may be damped by connecting the screws with elastic bands. Vertebral motion could be further damped by covering the anterior bands with rubber sleeves similar to the sleeves used over the posterior bands of the prior art devices described above.

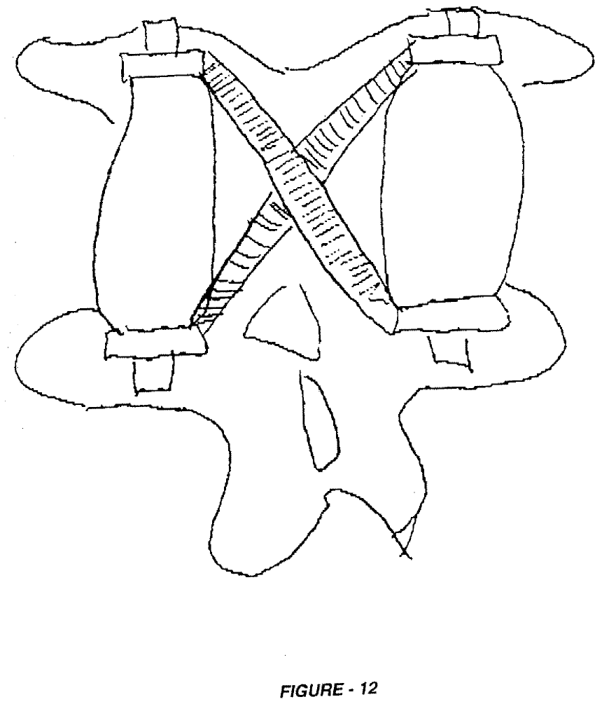

The cross-coupling elements according to the invention need not attach with pedicle screws. FIG. 12 illustrates an alternative configuration wherein the ends of the cross-coupling elements attached more directly to dampening elements. In addition, although in the preferred embodiment the cross-coupled elements attach at the points where the dampening elements attach, this is not essential to the invention, since the ends of the cross-coupling elements may attach at separate points while still providing resistance to twisting and/or rotational motion.

What is claimed is:

1. A method for stabilizing upper and lower spinal vertebrae having a disc space situated therebetween, comprising the steps of:
inserting first and second fasteners into the upper vertebra;
inserting third and fourth fasteners into the lower vertebra; and
connecting at least two of the first, second, third, and fourth fasteners with an elongate element.

2. The method of claim 1, wherein the first and third fasteners are substantially vertically aligned and the second and fourth fasteners are substantially vertically aligned.

3. The method of claim 1, wherein at least three of the first, second, third, and fourth fasteners are connected with the elongate element.

4. The method of claim 1, wherein the first, second, third, and fourth fasteners are connected with the elongate element.

5. The method of claim 1, wherein the elongate element is an elastic connector.

6. The method of claim 1, wherein the elongate element has a first end and a second end, and wherein the first and second ends are connected by a crimp.

7. The method of claim 1, wherein the elongate element is a cable.

8. The method of claim 1, wherein the elongate element is a band.

9. The method of claim 1, further comprising the step of inserting an object between the upper and lower spinal vertebrae that resists compression.

10. The method of claim 9, wherein the object is an intervertebral cage.

11. The method of claim 9, wherein the object is a distracting plug.

12. The method of claim 1, further comprising the step of inserting bone graft into the disc space between the upper and lower spinal vertebrae.

13. The method of claim 1, wherein the elongate element pulls at least two of the first, second, third, and fourth fasteners closer together.

14. The method of claim 1, wherein compression is applied across the disc space.

15. An apparatus for stabilizing upper and lower spinal vertebrae having a disc space situated therebetween, comprising:
first and second fasteners adapted for attachment to the upper vertebra;
third and fourth fasteners adapted for attachment to the lower vertebra; and
an elongate element interconnecting at least two of the first, second, third, and fourth fasteners.

16. The device of claim 15, further comprising an intervertebral cage adapted for placement into the disc space.

17. The device of claim 15, further comprising bone graft adapted for placement into the disc space.

18. The device of claim 15, wherein the elongate element is a band.

19. The device of claim 15, wherein the elongate element is a cable.

20. The device of claim 15, wherein the elongate element interconnects at least three of the first, second, third, and fourth fasteners.

21. The device of claim 15, wherein the elongate element interconnects the first, second, third, and fourth fasteners.

22. The device of claim 15, wherein each of the first, second, third, and fourth fasteners comprise a post adapted to be inserted into the upper or lower vertebrae and an enlarged element fitted to an end of the post that is adapted to engage the elongate element.

23. The device of claim 22, wherein the enlarged element further comprises a tunnel, a tube, or a groove that is adapted to engage the elongate element.
